(19) United States
(12) Patent Application Publication
(10) Pub. No.: US 2008/0234243 A1
(43) Pub. Date: Sep. 25, 2008

(54) METHOD OF TREATING AMYLOIDOSIS MEDIATED DISEASES

(76) Inventors: Theresa M. LaVallee, Rockville, MD (US); Anthony M. Treston, Rockville, MD (US); Todd A. Hembrough, Damascus, MD (US)

Correspondence Address: KING & SPALDING LLP, 1180 PEACHTREE STREET, ATLANTA, GA, 30309-3521, US

(21) Appl. No.: 12/023,710

(22) Filed: Jan. 31, 2008

Related U.S. Application Data
(60) Provisional application No. 60/898,611, filed on Jan. 31, 2007.

(51) Int. Cl. A61K 31/56; A61P 25/28

(52) U.S. Cl. 514/178; 514/169; 514/177

(57) ABSTRACT

The compounds of the present invention can be used to inhibit formation of beta-amyloid protein. The present invention provides methods of preventing, delaying onset of, or treating diseases or conditions characterized or mediated by amyloidosis. In particular, the present invention is useful for preventing, delaying onset of, and treating Alzheimer's disease and related diseases causing dementia.

### CROSS-REFERENCE TO RELATED APPLICATIONS

This application claims the benefit of U.S. Provisional Patent Application No. 60/898,611, filed Jan. 31, 2007, the contents of which are incorporated herein by reference.

### FIELD OF THE INVENTION

The present invention relates to methods of treating diseases or conditions associated with or mediated by amyloidosis. More particularly, the present invention relates to the administration of 2-methoxyestradiol or derivatives of 2-methoxyestradiol to a patient suffering from Alzheimer's disease or related neural diseases. The present invention also relates to methods of preventing Alzheimer's disease or delaying the onset of Alzheimer's disease.

### BACKGROUND OF THE INVENTION

Amyloidosis is not a single disease entity, but rather a diverse group of progressive disease processes characterized by extracellular tissue deposits of a waxy, starch-like protein called amyloid, which accumulates in one or more organs or body systems. As the amyloid deposits build up, they begin to interfere with the normal function of the organ or body system. There are at least 15 different types of amyloidosis. The major forms are primary amyloidosis without known antecedent, secondary amyloidosis following some other condition, and hereditary amyloidosis.

Secondary amyloidosis occurs in people who have a chronic infection or inflammatory disease, such as tuberculosis, a bacterial infection called familial Mediterranean fever, bone infections (osteomyelitis), rheumatoid arthritis, inflammation of the small intestine (granulomatous ileitis), Hodgkin's disease, and leprosy.

Amyloid deposits typically contain three components. Amyloid protein fibrils, which account for about 90% of the amyloid material, comprise one of several different types of proteins. These proteins are capable of folding into so-called “beta-pleated” sheet fibrils, a unique protein configuration which exhibits binding sites for Congo red resulting in the unique staining properties of the amyloid protein. In addition, amyloid deposits are closely associated with the amyloid P (pentagonal) component (AP), a glycoprotein related to normal serum amyloid P (SAP), and with sulfated glycosaminoglycans (GAG), complex carbohydrates of connective tissue.

Many diseases of aging are based on or associated with amyloid-like proteins and are characterized, in part, by the buildup of extracellular deposits of amyloid or amyloid-like material that contribute to the pathogenesis, as well as the progression of the disease. These diseases include, but are not limited to, neurological disorders such as Alzheimer's disease (AD), including diseases or conditions characterized by a loss of cognitive memory capacity such as, for example, mild cognitive impairment (MCI), Lewy body dementia, Down's syndrome, hereditary cerebral hemorrhage with amyloidosis (Dutch type), and the Guam Parkinson-Dementia complex. Other diseases which are based on or associated with amyloid-like proteins such as progressive supranuclear palsy, multiple sclerosis, Creutzfeld Jacob disease, Parkinson's disease, HIV-related dementia, ALS (amyotropic lateral sclerosis), Adult Onset Diabetes, senile cardiac amyloidosis, endocrine tumors, and others, including macular degeneration.

Although pathogenesis of these diseases may be diverse, their characteristic deposits often contain many shared molecular constituents. To a significant degree, this may be attributable to the local activation of pro-inflammatory pathways, thereby leading to the concurrent deposition of activated complement components, acute phase reactants, immune modulators, and other inflammatory mediators (McGeer et al., 1994).

Alzheimer's disease (AD) is a neurological disorder primarily thought to be caused by amyloid plaques, an accumulation of abnormal deposit of proteins in the brain. The most frequent type of amyloid found in the brain of affected individuals is composed primarily of Aβ fibrils. Scientific evidence demonstrates that an increase in the production and accumulation of beta-amyloid protein in plaques leads to nerve cell death, which contributes to the development and progression of Alzheimer's disease. Loss of nerve cells in strategic brain areas, in turn, causes reduction in the neurotransmitters and impairment of memory. The proteins principally responsible for the plaque build up include amyloid precursor protein (APP) and two presenilins (presenilin I and presenilin II). Sequential cleavage of the amyloid precursor protein (APP), which is constitutively expressed and catabolized in most cells, by the enzymes β and γ secretase leads to the release of an 39 to 43 amino acid Aβ peptide. The degradation of APPs likely increases their propensity to aggregate in plaques. It is especially the Aβ(1-42) fragment that has a high propensity of building aggregates, due to two very hydrophobic amino acid residues at its C-terminus. The Aβ(1-42) fragment is therefore believed to be mainly involved and responsible for the initiation of neutritic plaque formation in Alzheimer's disease and to have, therefore, a high pathological potential.

The symptoms of Alzheimer's disease manifest slowly and the first symptom may only be mild forgetfulness. In this stage, individuals may forget recent events, activities, the names of familiar people or things and may not be able to solve simple math problems. As the disease progresses, symptoms are more easily noticed and become serious enough to cause people with Alzheimer's disease or their family members to seek medical help. Mid-stage symptoms of Alzheimer's disease include forgetting how to do simple tasks such as grooming, and problems develop with speaking, understanding, reading, or writing. Later stage Alzheimer's disease patients may become anxious or aggressive, may wander away from home and ultimately need total care.

Presently, the only definite way to diagnose Alzheimer's disease is to identify plaques and tangles in brain tissue in an autopsy after death of the individual. Therefore, doctors can only make a diagnosis of “possible” or “probable” Alzheimer's disease while the person is still alive. Using current methods, physicians can diagnose Alzheimer's disease correctly up to 90 percent of the time using several tools to diagnose “probable” Alzheimer's disease. Physicians ask questions about the person's general health, past medical problems, and the history of any difficulties the person has carrying out daily activities. Behavioral tests of memory, problem solving, attention, counting, and language provide information on cognitive degeneration and medical tests, such as tests of blood, urine, or spinal fluid, and brain scans can provide some further information.

The management of Alzheimer's disease consists of medication-based and non-medication based treatments. Treatments aimed at changing the underlying course of the disease (delaying or reversing the progression) have so far been largely unsuccessful. Medicines that restore the deficit (defect), or malfunctioning, in the chemical messengers of the nerve cells (neurotransmitters), such as the cholinesterase inhibitors (ChEIs), have been shown to improve symptoms. Medications are also available to address the psychiatric manifestations of Alzheimer's disease.

Cholinesterase inhibitors, such as tacrine and rivastigmine, are currently the only class of agents that are approved by the FDA for the treatment of Alzheimer's disease. These agents are medicines that restore the defect, or malfunctioning, in the chemical neurotransmission in the brain. ChEIs impede the enzymatic degradation of neurotransmitters, thereby increasing the amount of chemical messengers available to transmit the nerve signals in the brain.

For some people in the early and middle stages of the disease, the drugs tacrine (COGNEX®, Morris Plains, N.J.), donepezil (ARICEPT®, Tokyo, JP), rivastigmine (EXELON®, East Hanover, N.J.), or galantamine (REMINYL®, New Brunswick, N.J.) may help prevent some symptoms from becoming worse for a limited time. Another drug, memantine (NAMENDA®, New York, N.Y.), has been approved for treatment of moderate to severe Alzheimer's disease. Also, some medicines may help control behavioral symptoms of Alzheimer's disease, such as sleeplessness, agitation, wandering, anxiety, and depression. Treating these symptoms often makes patients more comfortable and makes their care easier for caregivers. Unfortunately, despite significant treatment advances showing that this class of agents is consistently better than a placebo, the disease continues to progress, and the average effect on mental functioning has only been modest. ChEIs also have side effects that include gastrointestinal dysfunction, liver toxicity and weight loss.

Advances in the understanding of the brain abnormalities that occur in Alzheimer's disease are hoped to provide the framework for new targets of treatment that are more focused on altering the course and development of the disease. Many compounds, including anti-inflammatory agents, are being actively investigated. Clinical trials using specific cyclooxygenase inhibitors (COX-2), such as rofecoxib and celecoxib, are also underway.

Other diseases that are based on or associated with the accumulation and deposit of amyloid-like protein are mild cognitive impairment, Lewy body dementia (LBD), amyotrophic lateral sclerosis (ALS), inclusion-body myositis (IBM) and macular degeneration, in particular, age-related macular degeneration (AMD).

Mild cognitive impairment (MCI) is a general term most commonly defined as a subtle but measurable memory disorder. A person with MCI experiences memory problems greater than normally expected with aging, but does not show other symptoms of dementia, such as impaired judgment or reasoning. MCI is a condition that frequently reflects a preclinical stage of AD.

The deposition of β-amyloid within the entorhinal cortex (EC) is believed to play a key role in the development of mild cognitive impairment (MCI) in the elderly. This is in line with the observation that the cerebrospinal fluid (CSF) Aβ(1-42) levels decline significantly once AD becomes clinically overt. In contrast to CSF-Aβ(1-42), CSF-tau levels are significantly increased in the MCI stage, and these values continue to be elevated thereafter, indicating that increased levels of CSF-tau may help in detecting MCI subjects who are predicted to develop AD.

Lewy body dementia (LBD) is a neurodegenerative disorder that can occur in persons older than 65 years of age, which typically causes symptoms of cognitive (thinking) impairment and abnormal behavioral changes. Symptoms can include cognitive impairment, neurological signs, sleep disorder, and autonomic failure. Cognitive impairment is the presenting feature of LBD in most cases. Patients have recurrent episodes of confusion that progressively worsen. The fluctuation in cognitive ability is often associated with shifting degrees of attention and alertness. Cognitive impairment and fluctuations of thinking may vary over minutes, hours, or days.

Lewy bodies are formed from phosphorylated and nonphosphorylated neurofilament proteins; they contain the synaptic protein alpha-synuclein as well as ubiquitin, which is involved in the elimination of damaged or abnormal proteins. In addition to Lewy Bodies, Lewy neurites, which are inclusion bodies in the cell processes of the nerve cells, may also be present. Amyloid plaques may form in the brains of patients afflicted with LBD, however they tend to be fewer in number than seen in patients with Alzheimer's disease. Neurofibrillary tangles, the other micropathological hallmark of AD, are not a main characteristic of LBD, but are frequently present in addition to amyloid plaques.

Amyotrophic lateral sclerosis (ALS) is characterized by degeneration of upper and lower motor neurons. In some ALS patients, dementia or aphasia may be present (ALS-D). The dementia is most commonly a frontotemporal dementia (FTD), and many of these cases have ubiquitin-positive, tau-negative inclusions in neurons of the dentate gyrus and superficial layers of the frontal and temporal lobes.

Inclusion-body myositis (IBM) is a crippling disease usually found in people over age 50, in which muscle fibers develop inflammation and begin to atrophy, but in which the brain is spared and patients retain their full intellect. Two enzymes involved in the production of amyloid-β protein were found to be increased inside the muscle cells of patients with this most common, progressive muscle disease of older people, in which amyloid-β is also increased.

What is needed is a method of treating a patient suffering from diseases associated with amyloidosis such as Alzheimer's disease.

### BRIEF DESCRIPTION OF DRAWINGS

FIG. 1 provides a graph showing a decrease in soluble AD levels in CB17 SCID mice treated with 2ME2. Mice (5 animals per group) were treated orally for 5 or 20 days with 300 mg/kg/day 2ME2, and soluble Aβ levels were measure in whole brain homogenates. (*P=0.012 vs. control)

### SUMMARY OF THE INVENTION

The present invention is a method of treating diseases mediated by amyloidosis, in particular, Alzheimer's disease and related diseases causing dementia. The method includes, but is not limited to, the administration of an effective amount of 2-methoxyestradiol or a 2-methoxyestradiol derivative as described in U.S. patent application Ser. No. 08/102,767, filed Aug. 6, 1993, now U.S. Pat. No. 5,504,074; U.S. patent application Ser. No. 09/641,327, filed Aug. 18, 2000; U.S. patent application Ser. No. 09/779,331, filed Feb. 8, 2001; U.S. patent application Ser. No. 09/933,894, filed Aug. 21, 2001; U.S. patent application Ser. No. 09/939,208, filed Aug. 24, 2001; U.S. patent application Ser. No. 10/789,471, filed Feb. 27, 2004; U.S. patent application Ser. No. 10/856,340, filed May 28, 2004; U.S. patent application Ser. No. 11/077,977, filed on Mar. 11, 2005; and U.S. patent application Ser. No. 11/489,263 filed Jul. 19, 2006, all of which are incorporated herein by reference in their entirety.

The present invention comprises a method of treating diseases or conditions associated with or mediated by amyloidosis, comprising administering to a human or animal in need of such treatment, an effective amount of a compound selected from one or more of the following:

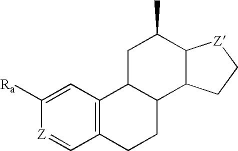

wherein Rₐ is selected from —OCH₃, —OCH₂CH₃, —CH₃, —CH₂CH₃, —CCCH₃, —CHCH—CH₃, or CH₂—CHCH₂; Z is selected from >C—OH, >C—F, >C—NH₂, >CCONH₂, >C—NHCOH, >C(H)—OSO₂NH₂, or >C—CHCH₂; and Z′ is selected from >C(H₂), >C(H)—CH₃, >C═CH₂, >C═CHCH₃ (cis or trans), >C═O, >C(H)—OH, >C(H)—O-alkyl or >C(H)—OSO₂NH₂. Alkyl is defined herein as a linear, branched and/or cyclic hydrocarbon chain containing 1-10 carbons.

In another embodiment, the present invention comprises a pharmaceutical preparation comprising a compound selected from one or more of the following:

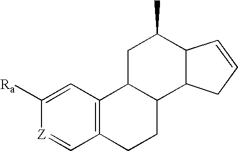

wherein Rₐ is selected from —OCH₃, —OCH₂CH₃, —CH₃, —CH₂CH₃, —CCCH₃, —CHCH—CH₃, or CH₂—CHCH₂; and Z is selected from >C—OH, >C—F, >C—NH₂, >CCONH₂, >C—NHCOH, >C(H)—OSO₂NH₂, or >C—CHCH₂. The pharmaceutical preparation can also comprise a pharmaceutically acceptable carrier, excipient or diluent.

### DETAILED DESCRIPTION

The present invention may be understood more readily by reference to the following detailed description of specific embodiments included herein. Although the present invention has been described with reference to specific details of certain embodiments thereof, it is not intended that such details should be regarded as limitations upon the scope of the invention. The entire text of the references mentioned herein are hereby incorporated in their entireties by reference.

As described below, compounds that are useful in accordance with the invention include 2-methoxyestradiol and 2-methoxyestradiol derivatives that are useful in treating or preventing conditions associated with amyloidosis and, in particular, Alzheimer's disease. 2-methoxyestradiol is an endogenous metabolite that is formed by the sequential hydroxylation of 17β-estradiol by cytochrome P450 followed by O-methylation by catechol-O-methyltransferase. 2-methoxyestradiol is capable of crossing the blood brain barrier and has several characterized mechanisms of action, one of which is to down regulate the expression of HIF-1 alpha (Mabjeesh et al. Cancer Cell, (2003) 3:363-75). HIF-1 is the primary transcription factor regulating oxygen homeostasis (Huang et al. J. Biol. Chem., (1999) 274:9038-9044). Hypoxia has been shown to play a role in progression of Alzheimer's Disease (Bazan et al. Mol. Neurobiol., (2002) 26(2-3):283-98). Further the gene expression of a β-secretase that processes APP and enables Aβ production, BACE-1, is responsive to regulatory control by HIF-1 (Sun et al. PNAS U.S.A., (2006) 103(49):18727-732). Without wishing to be bound by the following theory, 2-methoxyestradiol and/or 2-methoxyestradiol derivatives may be able to prevent or treat conditions associated with amyloidosis by inhibiting the formation of Aβ through one or more mechanisms of action, including the ability to regulate HIF-1.

Preferred compounds of the invention are 2-methoxyestradiol or 2-methoxyestradiol derivatives modified at the 2-, 3- or 17-positions or at combinations of the 2-, 3-, and 17-positions. Preferred compounds are those of the general Formulae I or II:

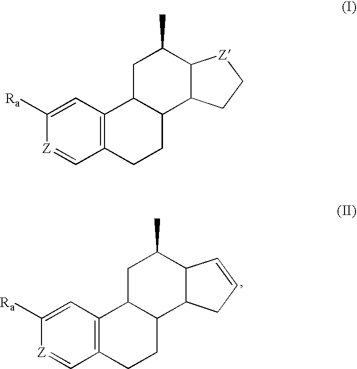

wherein Rₐ is selected from —OCH₃, —OCH₂CH₃, —CH₃, —CH₂CH₃, —CCCH₃, —CHCH—CH₃, or CH₂—CHCH₂; Z is selected from >C—OH, >C—F, >C—NH₂, >CCONH₂, >C—NHCOH, >C(H)—OSO₂NH₂, or >C—CHCH₂ and; Z′ is selected from >C(H₂), >C(H)—CH₃, >C═CH₂, >C═CHCH₃ (cis or trans), >C═O, >C(H)—OH, >C(H)—O-alkyl or >C(H)—OSO₂NH₂. Alkyl is defined herein as a linear, branched and/or cyclic hydrocarbon chain containing 1-10 carbons. Preferred species according to the present invention are described below.

In an alternate disclosed embodiment of the present invention, compounds according to the present invention are those of Formula I, wherein Rₐ is —OCH₃; Z is selected from >C—OH, >C—F, >C—NH₂, >CCONH₂, >C—NHCOH, >C(H)—OSO₂NH₂, or >C—CHCH₂; and Z′ is selected from >C(H₂), >C(H)—CH₃, >C═CH₂, >C═CHCH₃ (cis or trans), >C═O, >C(H)—OH, >C(H)—O-alkyl or >C(H)—OSO₂NH₂.

In another alternate disclosed embodiment of the present invention, compounds according to the present invention are those of Formula I, wherein Rₐ is —OCH₂CH₃; Z is selected from >C—OH, >C—F, >C—NH₂, >CCONH₂, >C—NHCOH, >C(H)—OSO₂NH₂, or >C—CHCH₂; and Z′ is selected from >C(H₂), >C(H)—CH₃, >C═CH₂, >C═CHCH₃ (cis or trans), >C═O, >C(H)—OH, >C(H)—O-alkyl or >C(H)—OSO₂NH₂.

In a further alternate disclosed embodiment of the present invention, compounds according to the present invention are those of Formula I, wherein Rₐ is —CH₃; Z is selected from >C—OH, >C—F, >C—NH₂, >CCONH₂, >C—NHCOH, >C(H)—OSO₂NH₂, or >C—CHCH₂; and Z′ is selected from >C(H₂), >C(H)—CH₃, >C═CH₂, >C═CHCH₃ (cis or trans), >C═O, >C(H)—OH, >C(H)—O-alkyl or >C(H)—OSO₂NH₂.

In a further alternate disclosed embodiment of the present invention, compounds according to the present invention are those of Formula I, wherein Rₐ is —CH₂CH₃; Z is selected from >C—OH, >C—F, >C—NH₂, >CCONH₂, >C—NHCOH, >C(H)—OSO₂NH₂, or >C—CHCH₂; and Z′ is selected from >C(H₂), >C(H)—CH₃, >C═CH₂, >C═CHCH₃ (cis or trans), >C═O, >C(H)—OH, >C(H)—O-alkyl or >C(H)—OSO₂NH₂.

In a further alternate disclosed embodiment of the present invention, compounds according to the present invention are those of Formula I, wherein Rₐ is —CCCH₃; Z is selected from >C—OH, >C—F, >C—NH₂, >CCONH₂, >C—NHCOH, >C(H)—OSO₂NH₂, or >C—CHCH₂; and Z′ is selected from >C(H₂), >C(H)—CH₃, >C═CH₂, >C═CHCH₃ (cis or trans), >C═O, >C(H)—OH, >C(H)—O-alkyl or >C(H)—OSO₂NH₂.

In a further alternate disclosed embodiment of the present invention, compounds according to the present invention are those of Formula I, wherein Rₐ is —CHCH—CH₃; Z is selected from >C—OH, >C—F, >C—NH₂, >CCONH₂, >C—NHCOH, >C(H)—OSO₂NH₂, or >C—CHCH₂; and Z′ is selected from >C(H₂), >C(H)—CH₃, >C═CH₂, >C═CHCH₃ (cis or trans), >C═O, >C(H)—OH, >C(H)—O-alkyl or >C(H)—OSO₂NH₂.

In a further alternate disclosed embodiment of the present invention, compounds according to the present invention are those of Formula I, wherein Rₐ is CH₂—CHCH₂; Z is selected from >C—OH, >C—F, >C—NH₂, >CCONH₂, >C—NHCOH, >C(H)—OSO₂NH₂, or >C—CHCH₂; and Z′ is selected from >C(H₂), >C(H)—CH₃, >C—CH₂, >C═CHCH₃ (cis or trans), >C═O, >C(H)—OH, >C(H)—O-alkyl or >C(H)—OSO₂NH₂.

In each of the cases where stereoisomers are possible, both R and S stereoisomers are envisioned as well as any mixture of stereoisomers.

In yet another alternate disclosed embodiment of the present invention, compounds according to the present invention are those of Formula II, wherein Rₐ is —OCH₃; and Z is selected from >C—OH, >C—F, >C—NH₂, >CCONH₂, >C—NHCOH, >C(H)—OSO₂NH₂, or >C—CHCH₂.

In another alternate disclosed embodiment of the present invention, compounds according to the present invention are those of Formula II, wherein Rₐ is —OCH₂CH₃; and Z is selected from >C—OH, >C—F, >C—NH₂, >CCONH₂, >C—NHCOH, >C(H)—OSO₂NH₂, or >C—CHCH₂.

In another alternate disclosed embodiment of the present invention, compounds according to the present invention are those of Formula II, wherein Rₐ is —CH₃; and Z is selected from >C—OH, >C—F, >C—NH₂, >CCONH₂, >C—NHCOH, >C(H)—OSO₂NH₂, or >C—CHCH₂.

In another alternate disclosed embodiment of the present invention, compounds according to the present invention are those of Formula II, wherein Rₐ is —CH₂CH₃; and Z is selected from >C—OH, >C—F, >C—NH₂, >CCONH₂, >C—NHCOH, >C(H)—OSO₂NH₂, or >C—CHCH₂.

In a further alternate disclosed embodiment of the present invention, compounds according to the present invention are those of Formula II, wherein Rₐ is —CCCH₃; and Z is selected from >C—OH, >C—F, >C—NH₂, >CCONH₂, >C—NHCOH, >C(H)—OSO₂NH₂, or >C—CHCH₂.

In another alternate disclosed embodiment of the present invention, compounds according to the present invention are those of Formula II, wherein Rₐ is —CHCH—CH₃; and Z is selected from >C—OH, >C—F, >C—NH₂, >CCONH₂, >C—NHCOH, >C(H)—OSO₂NH₂, or >C—CHCH₂.

In another alternate disclosed embodiment of the present invention, compounds according to the present invention are those of Formula II, wherein Rₐ is CH₂—CHCH₂; and Z is selected from >C—OH, >C—F, >C—NH₂, >CCONH₂, >C—NHCOH, >C(H)—OSO₂NH₂, or >C—CHCH₂.

Compounds that are useful in the present invention include, but are not limited to, the compounds of Table I.

TABLE I
3-Amines

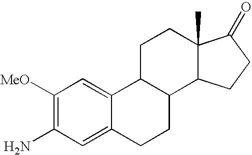

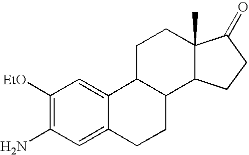

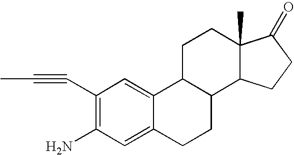

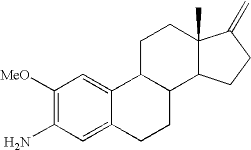

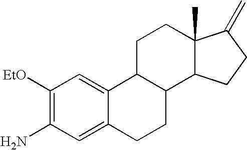

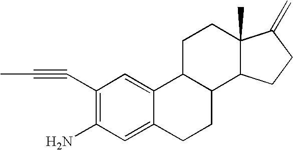

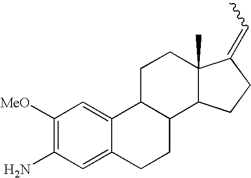

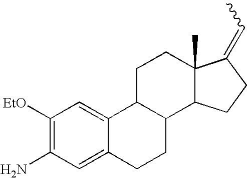

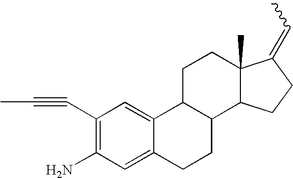

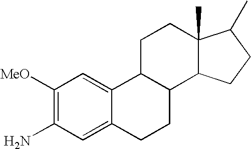

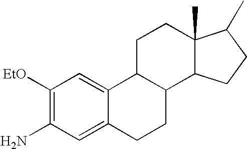

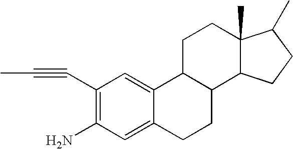

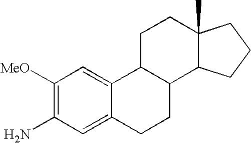

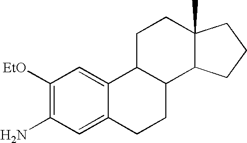

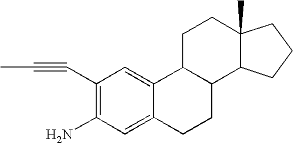

3-Carbamides

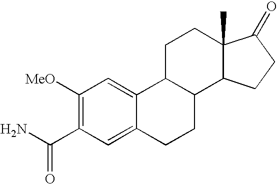

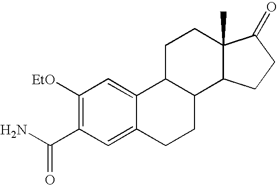

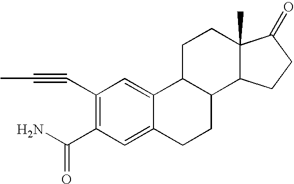

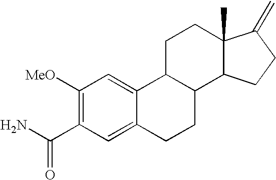

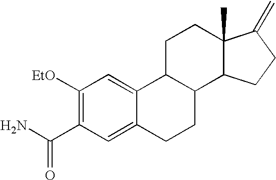

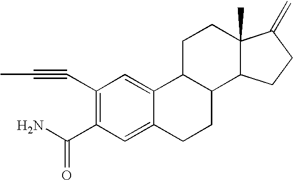

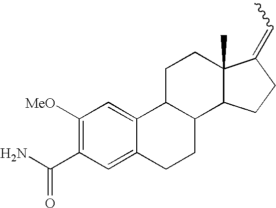

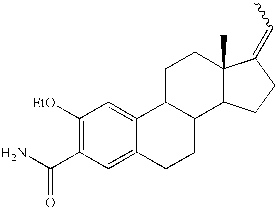

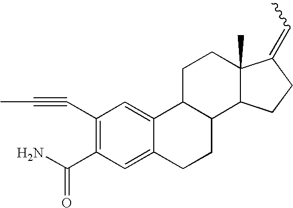

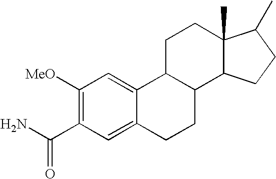

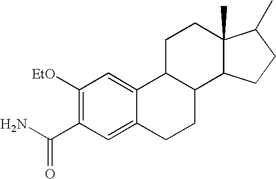

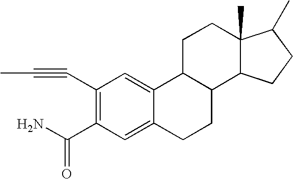

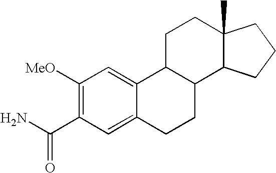

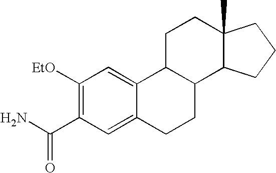

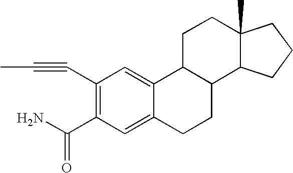

3-Formamides

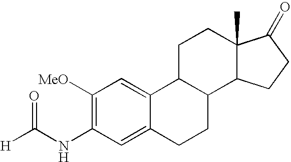

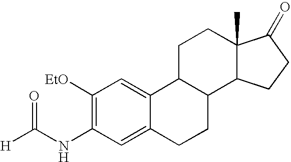

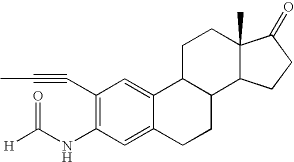

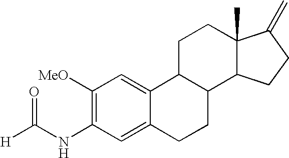

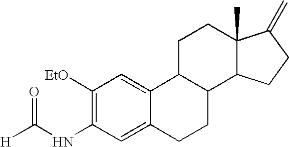

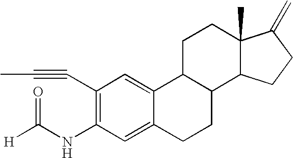

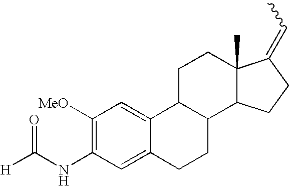

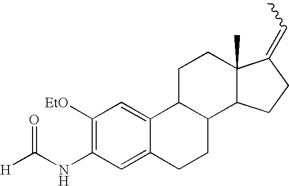

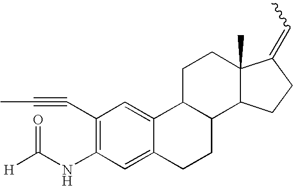

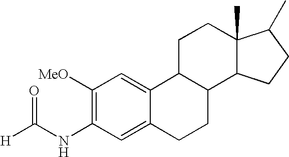

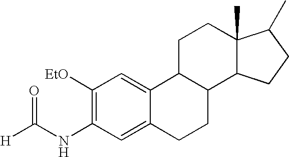

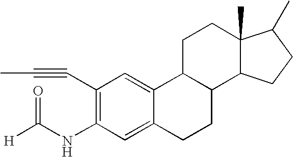

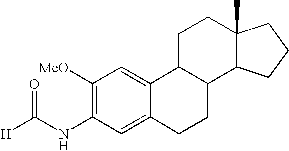

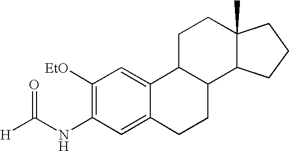

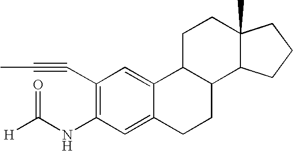

3-Sulfamates

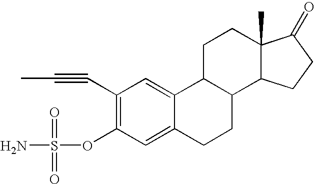

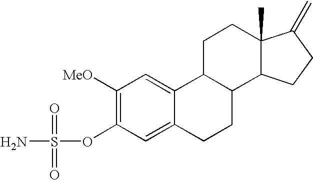

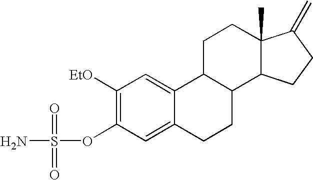

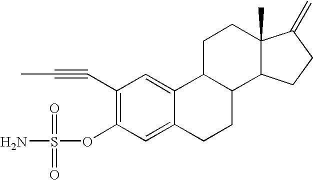

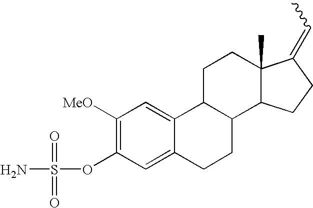

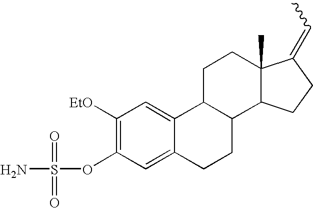

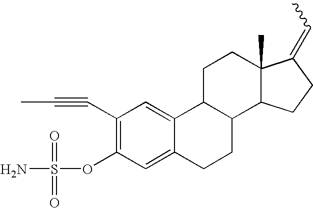

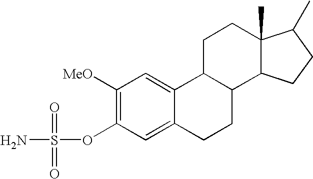

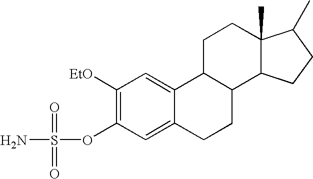

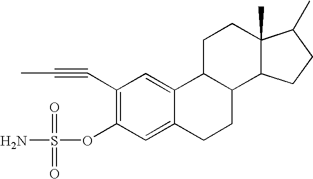

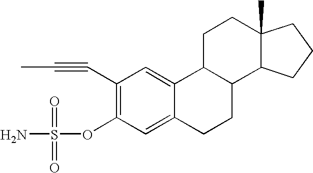

3-Vinyls

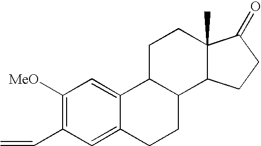

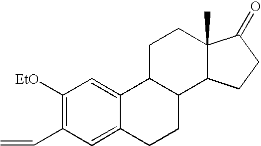

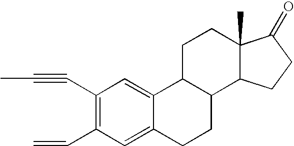

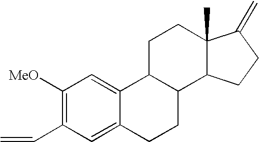

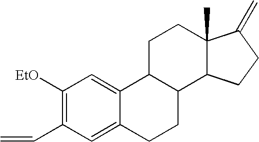

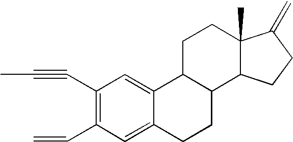

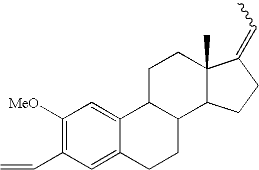

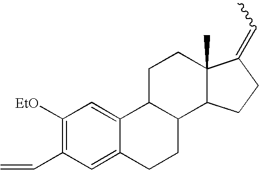

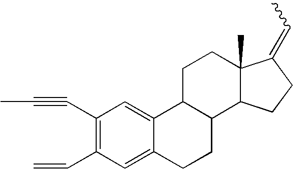

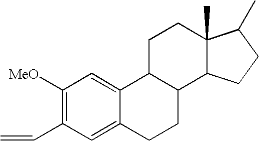

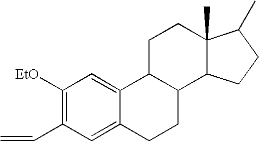

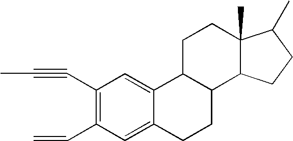

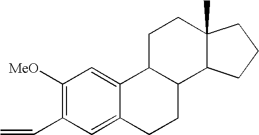

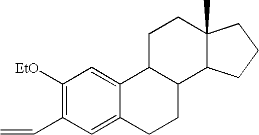

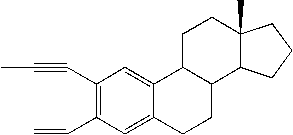

3-Fluoro

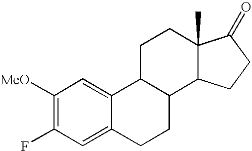

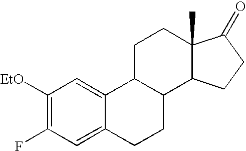

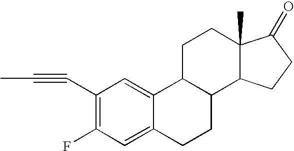

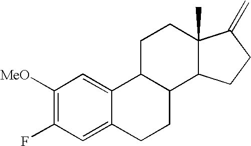

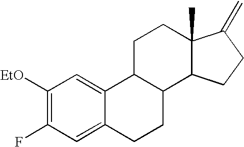

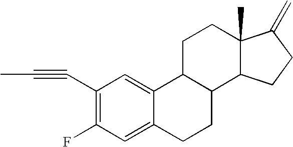

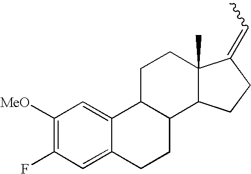

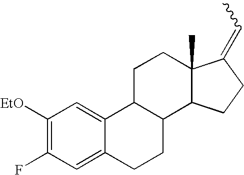

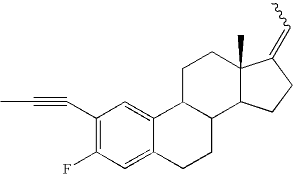

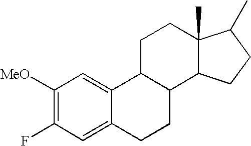

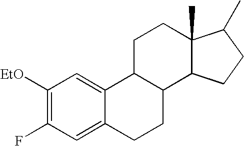

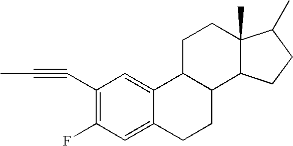

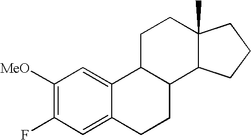

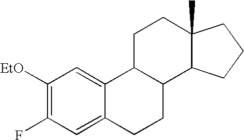

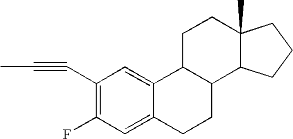

Those skilled in the art will appreciate that the invention extends to other compounds within the formulae provided above, having the described characteristics. These characteristics can be determined for each test compound using assays found known to those skilled in the art.

Also contemplated by the present invention are implants or other devices comprised of the compounds or drugs of Formulae I or II or prodrugs thereof where the drug or prodrug is formulated in a biodegradable or non-biodegradable polymer for sustained release. Non-biodegradable polymers release the drug in a controlled fashion through physical or mechanical processes without the polymer itself being degraded. Biodegradable polymers are designed to gradually be hydrolyzed or solubilized by natural processes in the body, allowing gradual release of the admixed drug or prodrug. The drug or prodrug can be chemically linked to the polymer or can be incorporated into the polymer by admixture. Both biodegradable and non-biodegradable polymers and the process by which drugs are incorporated into the polymers for controlled release are well known to those skilled in the art. Examples of such polymers can be found in many references, such as Brem et al., J. Neurosurg 74: pp. 441-446 (1991). These implants or devices can be implanted in the vicinity where delivery is desired, for example, at the site of a amyloid plaque deposition.

The present invention also relates to conjugated prodrugs and uses thereof. More particularly, the invention relates to conjugates of steroid compounds, such as compounds of Formulae I or II, and the use of such conjugates in the prophylaxis or treatment of conditions associated with amyloidosis and, in particular, Alzheimer's disease. The invention also relates to compositions including the prodrugs of the present invention and methods of synthesizing the prodrugs.

In one aspect, the present invention provides a conjugated prodrug of an estradiol compound, preferably compounds of Formulae I or II, conjugated to a biological activity modifying agent.

Alternatively, the conjugated prodrug according to the present invention includes the compounds of Formulae I or II conjugated to a peptide moiety.

The incorporation of an estradiol compound, such as the compounds of Formulae I or II, into a disease-dependently activated pro-drug enables significant improvement of potency and selectivity of this anti-amyloidosis agent.

In addition to the compounds of the present invention, the pharmaceutical composition of this invention may also contain, or be co-administered (simultaneously or sequentially) with, one or more pharmacological agents of value in treating one or more disease conditions referred to hereinabove.

A person skilled in the art will be able by reference to standard texts, such as Remington's Pharmaceutical Sciences 17th edition incorporated herein by reference, to determine how the formulations are to be made and how these may be administered.

In a further aspect of the present invention there is provided use of compounds of Formulae I or II or prodrugs thereof according to the present invention for the preparation of a medicament for the prophylaxis or treatment of conditions associated with amyloidosis and, in particular, Alzheimer's disease.

In a further aspect of the present invention there is provided a pharmaceutical composition comprising compounds of Formulae I or II or prodrugs thereof according to the present invention, together with a pharmaceutically acceptable carrier, diluent or excipient.

The pharmaceutical composition of the present invention may be used as a preventative agent or prophylactic to prevent or delay the onset of Alzheimer's disease or related neural diseases.

By “an effective amount” is meant a therapeutically or prophylactically effective amount. Such amounts can be readily determined by an appropriately skilled person, taking into account the condition to be treated, the route of administration and other relevant factors. Such a person will readily be able to determine a suitable dose, mode and frequency of administration.

Pharmaceutically acceptable salts of the compounds disclosed herein may be prepared in any conventional manner for example from the free base and acid. In vivo hydrolysable esters, amides and carbamates of the compounds disclosed herein may be prepared in any conventional manner and are considered to be included in the disclosed invention.

### Administration

The compositions described above can be provided as physiologically acceptable formulations using known techniques, and these formulations can be administered by standard routes. In general, the combinations may be administered by the topical, oral, rectal or parenteral (e.g., intravenous, subcutaneous or intramuscular) route. In addition, the combinations may be incorporated into polymers allowing for sustained release, the polymers being implanted in the vicinity of where delivery is desired, for example, at the site of a tumor or within or near the eye. The dosage of the composition will depend on the condition being treated, the particular derivative used, and other clinical factors such as weight and condition of the patient and the route of administration of the compound. However, for oral administration to humans, a dosage of 0.01 to 100 mg/kg/day.

The formulations in accordance with the present invention can be administered in the form of tablet, a capsule, a lozenge, a cachet, a solution, a suspension, an emulsion, a powder, an aerosol, a suppository, a spray, a pastille, an ointment, a cream, a paste, a foam, a gel, a tampon, a pessary, a granule, a bolus, a mouthwash, or a transdermal patch.

The formulations include those suitable for oral, rectal, nasal, inhalation, topical (including dermal, transdermal, buccal and sublingual), vaginal, parenteral (including subcutaneous, intramuscular, intravenous, intradermal, intraocular, intratracheal, and epidural) or inhalation administration. The formulations may conveniently be presented in unit dosage form and may be prepared by conventional pharmaceutical techniques. Such techniques include the step of bringing into association the active ingredient and a pharmaceutical carrier(s) or excipient(s). In general, the formulations are prepared by uniformly and intimately bringing into association the active ingredient with liquid carriers or finely divided solid carriers or both, and then, if necessary, shaping the product.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil emulsion, etc.

A tablet may be made by compression or molding, optionally with one or more accessory ingredients. Compressed tablets may be prepared by compressing, in a suitable machine, the active ingredient in a free-flowing form such as a powder or granules, optionally mixed with a binder, lubricant, inert diluent, preservative, surface-active or dispersing agent. Molded tablets may be made by molding, in a suitable machine, a mixture of the powdered compound moistened with an inert liquid diluent. The tablets may optionally be coated or scored and may be formulated so as to provide a slow or controlled release of the active ingredient therein.

Formulations suitable for topical administration in the mouth include lozenges comprising the ingredients in a flavored basis, usually sucrose and acacia or tragacanth; pastilles comprising the active ingredient in an inert basis such as gelatin and glycerin, or sucrose and acacia; and mouthwashes comprising the ingredient to be administered in a suitable liquid carrier.

Formulations suitable for topical administration to the skin may be presented as ointments, creams, gels and pastes comprising the ingredient to be administered in a pharmaceutical acceptable carrier. A preferred topical delivery system is a transdermal patch containing the ingredient to be administered.

Formulations for rectal administration may be presented as a suppository with a suitable base comprising, for example, cocoa butter or a salicylate.

Formulations suitable for nasal administration, wherein the carrier is a solid, include a coarse powder having a particle size, for example, in the range of 20 to 500 microns which is administered in the manner in which snuff is taken; i.e., by rapid inhalation through the nasal passage from a container of the powder held close up to the nose. Suitable formulations, wherein the carrier is a liquid, for administration, as for example, a nasal spray or as nasal drops, include aqueous or oily solutions of the active ingredient.

Formulations suitable for vaginal administration may be presented as pessaries, tampons, creams, gels, pastes, foams or spray formulations containing, in addition to the active ingredient, ingredients such as carriers as are known in the art to be appropriate.

Formulation suitable for inhalation may be presented as mists, dusts, powders or spray formulations containing, in addition to the active ingredient, ingredients such as carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in freeze-dried (lyophilized) conditions requiring only the addition of a sterile liquid carrier, for example, water for injections, immediately prior to use. Extemporaneous injection solutions and suspensions may be prepared from sterile powders, granules and tablets of the kinds previously described.

Preferred unit dosage formulations are those containing a daily dose or unit, daily sub-dose, as herein above recited, or an appropriate fraction thereof, of the administered ingredient.

It should be understood that in addition to the ingredients, particularly mentioned above, the formulations of the present invention may include other agents conventional in the art having regard to the type of formulation in question, for example, those suitable for oral administration may include flavoring agents.

The present invention includes compositions and methods for treating mammalian disease characterized by pathogenic amyloidosis by administering compounds of Formulae I or II. The 2-methoxyestradiol, and derivatives thereof, are modified at the 2-, 3- and 17-positions or combinations thereof. Combinations which are physically impossible are not contemplated by this invention, such as a carbon atom containing 5 bonds.

100% pure isomers are contemplated by this invention, however a stereochemical isomer (labeled as α or β, or as R or S) may be a mixture of both in any ratio, where it is chemically possible by one skilled in the art. Also contemplated by this invention are both classical and non-classical bioisosteric atom and substituent replacements, such as are described by Patani and Lavoie (“Bio-isosterism: a rational approach in drug design” Chem. Rev. (1996) p. 3147-3176) and are well known to one skilled in the art. Such bioisosteric replacements include, for example, but are not limited to, substitution of ═S or ═NH for ═O.

The compositions and methods are further illustrated by the following non-limiting examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention.

### EXAMPLE 1

### In Vivo Effects of 2-ME2 Analogs on the Presence and Concentration of Aβ in the Brains of CB.17 SCID Mice

CB. 17 SCID mice were treated orally for 5 or 20 days with 300 mg/kg/day 2ME2. At the end of treatment mice were humanely euthanized, and mouse brain was recovered surgically. This tissue was immediately frozen on dry ice until processing for ELISA analysis of soluble Aβ peptide. Serum is also routinely collected for later analysis as well.

### Preparation of the Brain Extract Solution

1. Homogenize brains into 15 vol of ice cold TBS, pH 7.4 containing leupeptide (10 ug/ml) and aprotinin (20 ug/ml).

2. Transfer to microcentrifuge tubes and spin 20,000 g for 20 min at 4° C.

3. Remove supernatant (TBS Extract) to ice.

4. Wash once with cold TBS and resuspend in 15 vol of TBS/1% Triton X-100 and protease inhibitors. Incubate 30 min at 4° C. with agitation.

5. Transfer to microcentrifuge tubes and spin 20,000 g for 20 min at 4° C.

6. Remove supernatant (Triton Extract) to ice.

7. Assay supernatants.

The various supernatants of the brain homogenate are assayed for the presence and concentration of murine amyloid beta peptide using the IBL Mouse/Rat Amyloidβ (1-40) ELISA Assay Kit (kit# 27730) following manufacturers protocol.

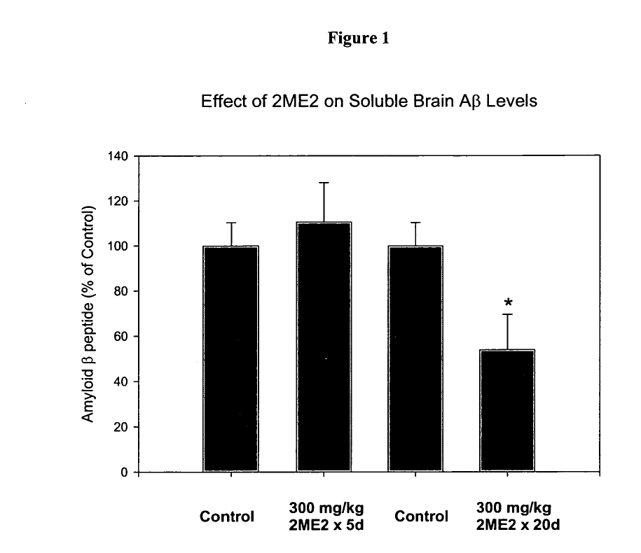

Harvested brains were homogenized with a tissue homogenizer, and subjected to serial extraction with TBS followed by TBS/Triton X-100. The resulting extracts were then assayed for the presence of the soluble Aβ (1-40) peptide. As expected, the majority of the soluble Aβ was present in the Triton X-100 fractions. In the first study, CB.17 SCID mice were treated orally with 300 mg/kg 2ME2 daily for 5 days. Brains were harvested, and soluble Aβ was assessed. In this study, (FIG. 1) no difference in soluble Aβ levels was seen between treated and control mice. In contrast, when CB17 SCID mice were treated orally with 300 mg/kg/day 2ME2 for 20 days, there was a statistically significant decrease (46% inhibition, P=0.012) in the levels of soluble Aβ. Comparable levels of soluble Aβ were seen in the control groups of the two studies.

We claim:

1. A method of treating diseases or conditions associated with or mediated by amyloidosis, comprising administering to a human or animal in need thereof, an effective amount of a compound having the structure: 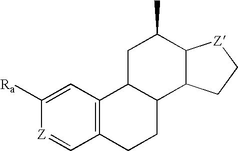
wherein Rₐ is selected from —OCH₃, —OCH₂CH₃, —CH₃, —CH₂CH₃, —CCCH₃, —CHCH—CH₃, or CH₂—CHCH₂; Z is selected from >C—OH, >C—F, >C—NH₂, >CCONH₂, >C—NHCOH, >C(H)—OSO₂NH₂, or >C—CHCH₂; and Z′ is selected from >C(H₂), >C(H)—CH₃, >C═CH₂, >C═CHCH₃ (cis or trans), >C═O, >C(H)—OH, >C(H)—O-alkyl or >C(H)—OSO₂NH₂.

2. The method of claim 1, wherein the disease or condition associated with or mediated by amyloidosis is selected from Alzheimer's Disease (AD), Lewy body dementia (LBD), amyotrophic lateral sclerosis (ALS), inclusion body myositis (IBM), or age-related macular degeneration (AMD).

3. The method of claim 1, wherein administration of the compound is oral, parenteral, transdermal, topical, intravenous, subcutaneous, intramuscular, intradermal, ophthalmic, epidural, intratracheal, sublingual, buccal, rectal, vaginal, nasal or inhalation.

4. The method of claim 3, wherein the compound further comprises a pharmaceutically acceptable carrier, diluent, or excipient.

5. A method of treating diseases or conditions associated with or mediated by amyloidosis, comprising administering to a human or animal in need thereof, an effective amount of a compound having the structure: 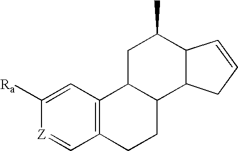
wherein Rₐ is selected from —OCH₃, —OCH₂CH₃, —CH₃, —CH₂CH₃, —CCCH₃, —CHCH—CH₃, or CH₂—CHCH₂; and Z is selected from >C—OH, >C—F, >C—NH₂, >CCONH₂, >C—NHCOH, >C(H)—OSO₂NH₂, or >C—CHCH₂.

6. The method of claim 5, wherein the disease or condition associated with or mediated by amyloidosis is selected from Alzheimer's Disease (AD), Lewy body dementia (LBD), amyotrophic lateral sclerosis (ALS), inclusion body myositis (IBM), or age-related macular degeneration (AMD).

7. The method of claim 5, wherein administration of the compound is oral, parenteral, transdermal, topical, intravenous, subcutaneous, intramuscular, intradermal, ophthalmic, epidural, intratracheal, sublingual, buccal, rectal, vaginal, nasal or inhalation.

8. The method of claim 7, wherein the compound further comprises a pharmaceutically acceptable carrier, diluent, or excipient.

9. A method of preventing or delaying onset of Alzheimer's disease, comprising administering to a human or animal in need thereof, an effective amount of a compound having the structure: 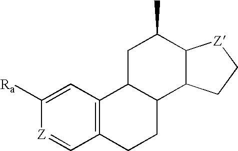
wherein Rₐ is selected from —OCH₃, —OCH₂CH₃, —CH₃, —CH₂CH₃, —CCCH₃, —CHCH—CH₃, or CH₂—CHCH₂; Z is selected from >C—OH, >C—F, >C—NH₂, >CCONH₂, >C—NHCOH, >C(H)—OSO₂NH₂, or >C—CHCH₂; and Z′ is selected from >C(H₂), >C(H)—CH₃, >C═CH₂, >C═CHCH₃ (cis or trans), >C═O, >C(H)—OH, >C(H)—O-alkyl or >C(H)—OSO₂NH₂.

10. The method of claim 9, wherein administration of the compound is oral, parenteral, transdermal, topical, intravenous, subcutaneous, intramuscular, intradermal, ophthalmic, epidural, intratracheal, sublingual, buccal, rectal, vaginal, nasal or inhalation.

11. The method of claim 10, wherein the compound further comprises a pharmaceutically acceptable carrier, diluent, or excipient.

12. A method of preventing or delaying onset of Alzheimer's disease, comprising administering to a human or animal in need thereof, an effective amount of a compound having the structure: 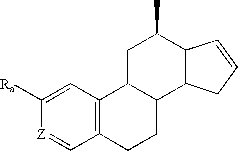
wherein Rₐ is selected from —OCH₃, —OCH₂CH₃, —CH₃, —CH₂CH₃, —CCCH₃, —CHCH—CH₃, or CH₂—CHCH₂; and Z is selected from >C—OH, >C—F, >C—NH₂, >CCONH₂, >C—NHCOH, >C(H)—OSO₂NH₂, or >C—CHCH₂.

13. The method of claim 12, wherein administration of the compound is oral, parenteral, transdermal, topical, intravenous, subcutaneous, intramuscular, intradermal, ophthalmic, epidural, intratracheal, sublingual, buccal, rectal, vaginal, nasal or inhalation.

14. The method of claim 13, wherein the compound further comprises a pharmaceutically acceptable carrier, diluent, or excipient.

15. A method of inhibiting formation of beta-amyloid (Aβ) protein, comprising administering to a human or animal an effective amount of a compound having the structure: 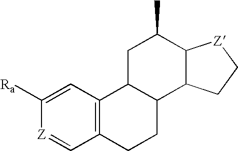
wherein Rₐ is selected from —OCH₃, —OCH₂CH₃, —CH₃, —CH₂CH₃, —CCCH₃, —CHCH—CH₃, or CH₂—CHCH₂; Z is selected from >C—OH, >C—F, >C—NH₂, >CCONH₂, >C—NHCOH, >C(H)—OSO₂NH₂, or >C—CHCH₂; and Z′ is selected from >C(H₂), >C(H)—CH₃, >C═CH₂, >C═CHCH₃ (cis or trans), >C═O, >C(H)—OH, >C(H)—O-alkyl or >C(H)—OSO₂NH₂.

16. The method of claim 15, wherein administration of the compound is oral, parenteral, transdermal, topical, intravenous, subcutaneous, intramuscular, intradermal, ophthalmic, epidural, intratracheal, sublingual, buccal, rectal, vaginal, nasal or inhalation.

17. The method of claim 16, wherein the compound further comprises a pharmaceutically acceptable carrier, diluent, or excipient.

18. A method of inhibiting formation of beta-amyloid (Aβ) protein, comprising administering to a human or animal an effective amount of a compound having the structure: 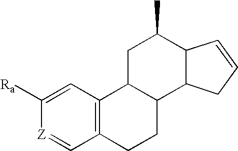
wherein Rₐ is selected from —OCH₃, —OCH₂CH₃, —CH₃, —CH₂CH₃, —CCCH₃, —CHCH—CH₃, or CH₂—CHCH₂; and Z is selected from >C—OH, >C—F, >C—NH₂, >CCONH₂, >C—NHCOH, >C(H)—OSO₂NH₂, or >C—CHCH₂.

19. The method of claim 18, wherein administration of the compound is oral, parenteral, transdermal, topical, intravenous, subcutaneous, intramuscular, intradermal, ophthalmic, epidural, intratracheal, sublingual, buccal, rectal, vaginal, nasal or inhalation.

20. The method of claim 19, wherein the compound further comprises a pharmaceutically acceptable carrier, diluent, or excipient.
